SEQUENCE LISTING (4 sequences)

SEQ ID NO: 1 - DNA, 702 bp, Artificial sequence
  Fibrino(gen)lytic Gene No. 2
aaaagagaggctgaagctaatcgtactcctgaacaacaaatctatgacccctacaaatacgttgagactgtctttgttgtggacaaagcaatggtcacaaaatacaatggcgatttagataagataaaaacaagaatgtacgaagctgccaacaatatgaatgagatgtacagatatatgttttttcgtgtagtaatggttggcctaataatttggaccgaagaagataagattaccgtgaagccagatgtggattatactttgaacgcatttgcagaatggagaaaaacatatttgctggctgagaaaaaacatgataatgctcagttaatcacgggcattgacttcagaggaagcattataggatacgcttacattggcagcatgtgccacccgaagcgttctgtaggaattattcaggattatagcccaataaatcttgtgcttgccgttataatggcccatgagatgggtcacaatctgggcattcaccatgacgacggttactgttattgcggtggttacccatgcattatgggtccctcgataagccctgaaccttccaaatttttcagcaattgtagttatatccaatgttgggactttattatgaatcacaacccagaatgcattgacaatgaacccttgggaacagatattatttcacctccactttgtggaaatgaacttttggaggcgtga

SEQ ID NO: 2 - DNA, 31 bp, Artificial sequence
  Primer 1
agagaggctgaagctaatcttactcctgaac

SEQ ID NO: 3 - DNA, 30 bp, Artificial sequence
  Primer 2
ctctcgagaaaagagaggctgaagctaatc

SEQ ID NO: 4 - DNA, 29 bp, Artificial sequence
  Primer 3
gagcggccgcctcacgcctccaaaagttc

(19) United States
(12) Patent Application Publication
(10) Pub. No.: US 2008/0312170 A1
(43) Pub. Date: Dec. 18, 2008

(54) Fibrinolysin of Agkistrodon Acutus Venom and its Usage

(76) Inventors: Guangmei Yan, Guangzhou (CN); Jiashu Chen, Guangzhou (CN); Pengxin Qiu, Guangzhou (CN); Hong Shan, Guangzhou (CN)

Correspondence Address: BRINKS HOFER GILSON & LIONE, P.O. BOX 10395, CHICAGO, IL, 60610, US

(21) Appl. No.: 11/793,071

(22) PCT Filed: Dec. 15, 2004
(86) PCT No.: PCT/CN04/01457

§ 371 Date: Apr. 8, 2008

(51) Int. Cl. A61K 31/70; C12N 15/11; C12N 15/00; A61P 7/00; C12N 1/19

(52) U.S. Cl. 514/44; 536/23.5; 435/320.1; 435/254.2

(57) ABSTRACT

The present invention relates to a fibrinolysin gene of Agkistrodon acutus, a vector containing the said gene and the host cell which transformed by the vector. The fibrinolysin could be used in treating diseases caused by thrombus. The fibrinolysin FII is purified from the crude venom of Agkistrodon acutus, and the corresponding gene is cloned. It is recommended to produce the fibrionolysin by the yeast expression system. The activity of the fibrionlysin is also determined. The advantages of this invention are that the expression level and activity of the fibrinolysin are both high and the quality of the fibrinolysin is stable.

### BACKGROUND OF THE INVENTION

The present invention relates to technology field of genetic engineering. Particularly, the present invention relates to the fibrino(gen)lytic gene No. 2 from Agikistrodon acutus, the gene vector, the genetic engineering host cell using the gene vector and the drugs prepared by the gene to counteract the thromboemolic disorders.

At present, thromboemolic disorders, especially myocardial infarction and apoplexy have become one of the main causes of mortality and morbidity for human beings in the world. With the successful control over the most infectious diseases, the improvement of people's living standard, the prevalent increase of people's life-span and the aging of the population structure in China, the morbidity caused by cardiovascular and cerebrovascular thromboemolic disorders has ranked the first and second places among all the diseases, respectively. It is estimated that there are at least 3 million cases annually. It is reasonably expected that the morbidity caused by thromboemolic disorders would be even higher in the future.

Nowadays, the drugs for treating thromboemolic disorders mainly include antiplatelet agents (for example, aspirin), anticoagulant (for example, heparin) and thrombolytic agents (for example, streptokinase and urokinase). Antiplatelet and anticoagulant agents, such as aspirin and heparin, enhance the effects of thrombolysis by preventing new fibrin formation. However they have no effect on existing thrombus. The clinically available thrombolytic agents, such as Streptokinase, urokinase and tissue plasminogen activator (t-PA), are plasminogen activators and effectively dissolve the thrombus by activating plasminogen. That is, the common characteristics of the thrombolytic agents is the indirect, slow and weak thrombolytic effect, especially when the thrombolytic agents act on larger thrumbosis. Where cardiovascular and cerebrovascular thrombosis occur, myocardium and neuron will die owing to hypoxia in few minutes after the blood stream is cut off. Due to their indirect thrombolysis mechanism, the above-mentioned three drugs have some side effects, such as lower selectivity with thrombosis and hemorrhage. Moreover, their broad application is limited because staphylokinase can induce anaphylaxis, and tissue plasminogen activator is also expensive.

It should be especially noted that thrombolytic agents are ineffective for about 25% patients even though combinated with other agents. Moreover, reocclusion occurs in about 5%-30% of the initially successful cases treated with thrombolytic agents, which has no susceptive on the thrombolytic agents.

In conclusion, the thrombolytic agents can not completely meet the requirements of the clinical application. It is urgent to develop a more specific and efficient thrombolytic drugs.

Foreign studies on snake venom show that the venom of Agkistrodon contortrix and Crotalus atrox (western diamondback rattlesnake) contains directly-fibrinolytic enzymes without activating plasminogen, which have showed thrombolytic activities in the venous thrombolysis model in rat. Additionally, hemorrhage and putrescence were not observed by microscopic examination of tissue sections from heart, liver, lung and kidney.

The thrombin-like enzymes isolated from some snake venom used in clinic in China, such as ancord, ahylysantinfarctase, is to convert fibrinogen into fibrin derivative and lower plasma viscosity to achieve antithrombotic effect with the similar mechanism as thrombin. Their efficacy and mechanism can not be compared with thrombolytic agents at all.

The unique venom of Agikistrodon acutus in China contains components that can directly degrade fibrin/fibrinogen and have a strong biological activity. Several fibrinolytic proteins are obtained from 15 aliquots by using column chromatography purification methods, among which Fraction II possesses biological characteristics of high value: (1) Direct thrombolysis: the fibrinolytic factors can degrade fibrin after the plasminogen is inactivated by hot plate methods; (2) Quick action: In in vitro experiments, the action of fibrinolytic factor is twice as fast as urokinase; (3) High efficiency: biological activity of fibrinolytic factor is higher than that of urokinase (130 μg fibrinolytic factor is equivalent to 450 u urokinase); (4) Low side effects: Although not-purified venom of Agikistrodon acutus has obvious side effect of bleeding, hemorrhage are not observed at the dosage of 500 μg/ml fibrinolytic factor. These strongly support that the fibrinolytic factor from the venom of Agikistrodon acutus may be a new generation of agent for treating thromboemolic disorders.

However, there are regional and seasonal diversities in the composition and biological activity of the venom of Agikistrodon acutus from natural source. In fact it is difficult to achieve steady clinical effect. Furthermore, owing to the complexity of crude snake components, it is still difficult to get a single component even after purification, which may lead to some toxicity and side effects in clinical application. Natural snake venom has the characteristics of limited production, high costs and low market penetration capacity. Its molecular structure and the relationship between structure and activity still can not be interpreted with protein technology. Therefore, solving these above-mentioned issues is necessary to establish the scientific base of the clinical application of the fibrinolytic factor from the venom of Agikistrodon acutus.

Yeast can grow fast and be bulk fermented without special culture medium. The yeast's DNA is relatively simple and convenient for cloning screening. Yeast is a fungus, so the mechanism of gene expression and regulation and processing modification of expression product are more complicated than those of E. Coli. For example, yeast can glycosylate and form the correct disulfide bonds, etc. There have been some precedents that snake venom protein successfully expressed in yeast.

In the present invention, using the antibody of FII from Agkistrodon acutus as a probe, the inventors screened the gene of FII from cDNA library and cloned to the yeast expression vector PPIC9K, then transformed into the yeast cell and achieved high expression level of FII from Agkistrodon acutus in yeast cell. At last the inventors purified the recombinant FII from Agkistrodon acutus by Chromatography and determined its Fibrinolytic activity. Abundance recombinant FII from Agkistrodon acutus by genetic engineering was obtained in the study, which lays the foundation for the follow-up pharmacodynamics study and the evaluation of any class I new drug.

Snake venom research has played a key role in the development of the biomedicine, such as isolation of DNA endonuclease from the snake venom which has promoted the study of molecular biology greatly, the discovery of toxins from Coral snake, which played a crucial role in the purification of neurotransmitter receptors. Compared with all kinds of biochemical clinical agents from snake venoms, recombinant FII from the venom of Agkistrodon acutus in the invention can overcome regional diversities and chemical heterogeneity in the composition and biological activity, increase production and efficacy, and become a new potential clinical drug with huge market value and intellectual property rights.

### SUMMARY OF THE INVENTION

An isolated polynucleotide (No.2 gene) comprises a sequence selected from a group consisting of (A) A polynucleotide having the following sequence:

5′-aaaagagaggctgaagctaatcgtactcctgaacaacaaatctatga
cccctacaaatacgttgagactgtctttgttgtggacaaagcaatggtca
caaaatacaatggcgatttagataagataaaaacaagaatgtacgaagct
gccaacaatatgaatgagatgtacagatatatgttttttcgtgtagtaat
ggttggcctaataatttggaccgaagaagataagattaccgtgaagccag
atgtggattatactttgaacgcatttgcagaatggagaaaaacatatttg
ctggctgagaaaaaacatgataatgctcagttaatcacgggcattgactt
cagaggaagcattataggatacgcttacattggcagcatgtgccacccga
agcgttctgtaggaattattcaggattatagcccaataaatcttgtgctt
gccgttataatggcccatgagatgggtcacaatctgggcattcaccatga
cgacggttactgttattgcggtggttacccatgcattatgggtccctcga
taagccctgaaccttccaaatttttcagcaattgtagttatatccaatgt
tgggactttattatgaatcacaacccagaatgcattgacaatgaaccctt
gggaacagatattatttcacctccactttgtggaaatgaacttttggagg
cgtga-3′;

(B) A polynucleotide sequence that is at least 80% identical to the polynucleotide sequence of (A); and (C) A fragment of (A) or (B).

### BRIEF DESCRIPTION OF THE DRAWINGS

FIG. 1 is the DEAE-SephadexA-50 ion exchange chromatograph of Agkistrodon acutus snake venom.

FIG. 2 is the first SephadexG-75 gel filtration of FII.

FIG. 3 is the second SephadexG-75 gel filtration of FII.

FIG. 4 illustrates the determination of the molecular weight of FII by SDS-PAGE.

FIG. 5 illustrates the standard curve of Protein Markers by SDS-PAGE.

FIG. 6 is the analysis of the degradation products of fibrinogen hydrolysis by SDS-PAGE.

FIG. 7 illustrates the fibrinolytic activity of FII.

FIG. 8 is the analysis of the total RNA of Agkistrodon acutus venom by Agarose-Formaldehyde Electrophoresis.

FIG. 9 illustrates the stucture of cDNA library of Agkistrodon acutus venom.

FIG. 10 illustrates the positive results of FII in the cDNA library of Agkistrodon acutus venom screened by monoclonal antibodies.

FIG. 11 is the nine positive clones' insertion DNA fraction sequences.

FIG. 12 is the FII amino acid sequence deduced by the ORF DNA sequence of FII.

FIG. 13 is the analysis of FII fusion Protein expressing in E.coil by SDS-PAGE.

FIG. 14 illustrates the two steps in constructing the target DNA-vector of FII gene in E.coil.

FIG. 15 illustrates the diploid restrictions in the plasmid pPIC9K-FII.

FIG. 16 illustrates the fibrinolytic activity of FII in the supernatant by SDS-PAGE.

FIG. 17 is the DEAE-Sepharose icon exchange chromatography of the supernatant.

FIG. 18 is the Buty-Toyopearl chromatography of the supernatant.

FIG. 19 illustrates the determination of the recombinant FII purity by SDS-PAGE.

FIG. 20 is the directly fibrinolytic activity of recombinant FII.

FIG. 21 illustrates the effect on IgG and albumin of recombinant FII by SDS-PAGE.

FIG. 22 illustrates the effect of recombinant FII on the Beagle's pulmonary artery thrombosis model.

### DETAILED DESCRIPTION INVENTION

Fibrinolytic enzyme FII from Agkistrodon acutus can directly degrade fibrin. Hemorrhage is not observed at the dosage of the effect fibrinolytic activity. However, the components of crude snake venom are complex and difficult to get a single component even after purification. It isn't conducive to large-scale production because of the limited output of Snake venom. The above-mentioned issues can be resolved through expressing recombinant fibrinolytic enzyme FII from Agkistrodon acutus by genetic engineering technology. The inventors isolates FII, a fibrinolytic enzyme from the venom of Agkistrodon acutus, achieves the screening and cloning of the FII gene, then expressed the recombinant fibrinolytic enzyme FII in yeast cell, and purified it and tested its fibrinolytic activity.

### EXAMPLES

### Experimental:

Methods: the natural fibrinolytic enzyme FII is isolated from Agkistrodon acutus venom by ion exchange chromatography and gel filtration. Its fibrinolytic activity is determinated by fibrin and fibrinogen used as substrate. Its reacting site on fibrinogen is observed by SDS-PAGE. Polyclonal antibody, monoclonal antibodies and cDNA library of Agkistrodon acutus venom are prepared. The gene of FII from cDNA library is screened by using the antibody of FII as a probe, which is cloned to the yeast expression vector PPIC9K by digestion and connection, yeast strains of high expression is obtained by transformation and screening. The recombination FII is purified by ion exchange chromatography and hydrophobic chromatography. Its purity determination is tested by SDS-PAGE. Fibrinolytic activity is determined by fibrin plate method. The effect on IgG and albumin is observed by SDS-PAGE. And the fibrinolytic activity in vivo is determined by Beagle's pulmonary artery thrombosis model.

Result: with a three-step procedure, the inventors obtained a fibrinolytic enzyme FII, which appears as a single band on SDS-PAGE and the molecular weight is 25,500. FII degraded, primarily, the Aα Bβ chains of fibrinogen and fibrin, while the γ chain was minimally affected. FII degraded fibrin in a dose-dependent manner. Through preparation of polyclonal antibody, monoclonal antibodies and cDNA library, the inventors screened the gene FII from the cDNA library, constructed recombinant plasmid, PPIC9K-FII. After transformation, recombinant plasmid was inserted into yeast's chromosome determined by PCR. Using fibrinolytic activity as a standard, the inventors screened a high expression strain, and the fibrinolytic activity reached a peak on the third days of expression. With a two-step procedure, the inventors obtained recombinant fibrinolytic enzyme FII, which appears as a single band on SDS-PAGE. Recombinant FII can strongly degrade fibrin, while have no effect on IgG and albumin. Recombinant FII can efficiently dissolve the preformed thrombus in the right lower Pulmonary artery when measurements made at 1 hours after recombinant FII infusion. The average of the recanalization rate of thrombosis of the right lower Pulmonary artery was 83.3%.

Conclusion: recombinant fibrinolytic enzyme FII is highly expressed in yeast. Recombinant FII has high fibrinolytic activity.

Natural FII from Agkistrodon acutus snake venom has low yield, instable quality. The inventors expressed recombinant FII in yeast by genetic engineering technology. The recombinant FII has higher fibrinolytic activity, more stable quality, higher output and lower cost than nature FII. The recombinant FII can dissolve effectively thrombus in animal thrombolysis models, and little hemorrhage is observed. It's a strong suggestion that the recombinant FII will become a novel and effective thrombolytic agent that has tremendous benefits on both social and economic fields.

### EXAMPLE 1

Purification of FII from Agkistrodon acutus snake venom DEAE-Sephadex A-50 ion exchange chromatography.

Method: DEAE-Sephadex A-50 was washed three times in 150 ml 0.05 M ammonium acetate (pH 8.0) and doused in the same buffer at 25° C. for 24 hours. The wet-washed DEAE-Sephadex A-50 was poured in a column (2.6 cm×100 cm) from the top until the gel layer was within one inch of the upper end of the column, then equilibrated with 0.05 M ammonium acetate (PH 8.0) at 25° C. for 24 hours. Agkistrodon acutus snake venom (3 g) was dissolved in 10 mL 0.05 M ammonium acetate (pH 8.0) and centrifuged at 1,500 rpm for 15 min, and the supernatant was applied to the column of DEAE-Sephadex A-50. Fractions were eluted with a linear gradient consisting of 0.05 M ammonium acetate (pH 8.0) as starting buffer and 1 M ammonium acetate (pH 5.0) as limit buffer. A volume of 3 mL fraction was collected at a flow rate of 12 mL/h. The absorbance of eluate was tested at 280 nm. Fibrinolytic activity was tested for peak fractions. Then the fibrinolytic fraction was dialyzed and lyophilized.

Fibrinolytic activity was demonstrated by using a modified fibrin plate technique (Astrup and Mullerti, 1952). Each concentration was tested three times. Bovine plasma (0.2%, 20 mL) was clotted in a Petri dish (diameter, 9.5 cm) with thrombin (80 μL, 100 U/mL). Fribrinolytic activity was expressed as the diameter product (mm2) of the lysed zone caused by 20 μL of each fraction. Each fraction was put on the surface of the dish and incubated at 37° C. for 12 hours.

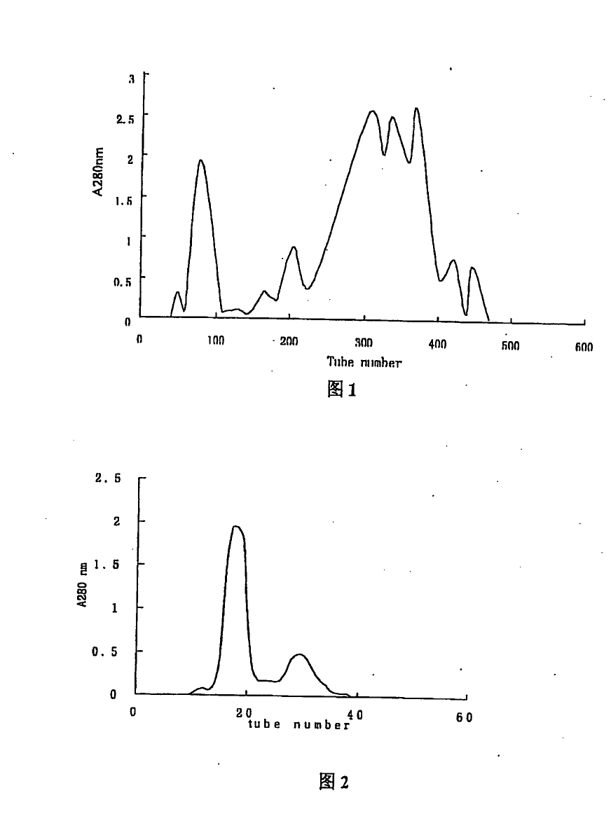

Results: Ion exchange chromatography of crude venom on DEAE-Sephadex A-50 yielded ten fractions (FIG. 1). The second fraction (FII) had higher fibrinolytic activity. Its fibrinolytic activity was 60.23±16.47 mm²/μg.

The first sephadex G-75 gel filtration Method: Sephadex G-75 was washed three times in 150 ml 0.05 M ammonium acetate(pH 8.0) and doused in the same buffer at 25° C. for 24 hours. The wet-washed Sephadex G-75 was poured in a column (1.1 cm×100 cm) from the top until the gel layer was within one inch of the upper end of the column, then equilibrated with 0.05 M ammonium acetate (PH 8.0) at 25° C. for 24 hours. FII (85 mg) obtained from DEAE-Sephadex A-50 ion exchange chromatography was dissolved in 2 mL 0.05M ammonium acetate (pH 8.0), and the supernatant was applied to the column of Sephadex G-75. Fractions were eluted with 0.05 M ammonium acetate (pH 8.0). A volume of 3 mL fraction was collected at a flow rate of 12 mL/hour. The absorbance of eluate was tested at 280 nm. The peak fractions tested fibrinolytic activity. Then the fibrinolytic fraction was dialyzed and lyophilized.

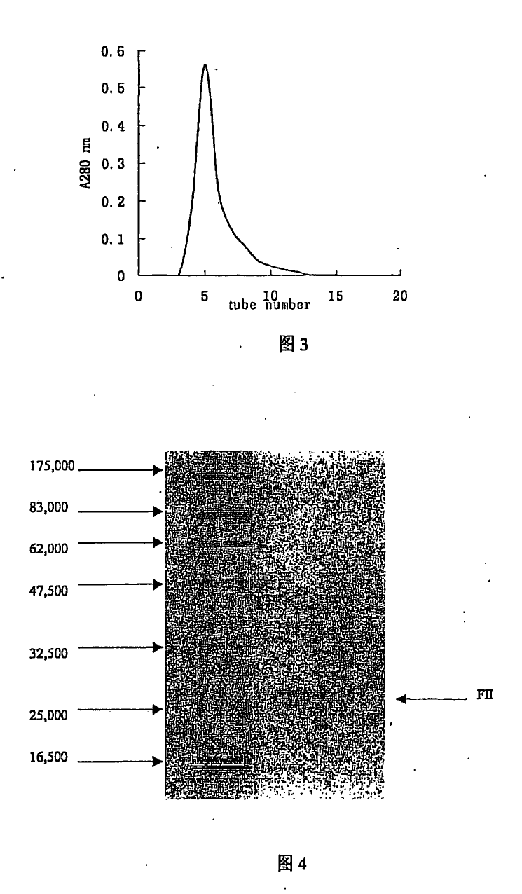

Results: After further fractionation by gel filtration on a Sephadex G-75 column, two fractions of FII were obtained (FIG. 2). The fibrinolytic activity was localized in the first fraction. Its fibrinolytic activity was 87.51±24.95 mm²/μg.

1.3 The second Sephadex G-75 gel filtration.

Method: The fibrinolytic fraction from the first Sephadex G-75 gel filtration was again applied to Sephadex G-75 gel column by the above method.

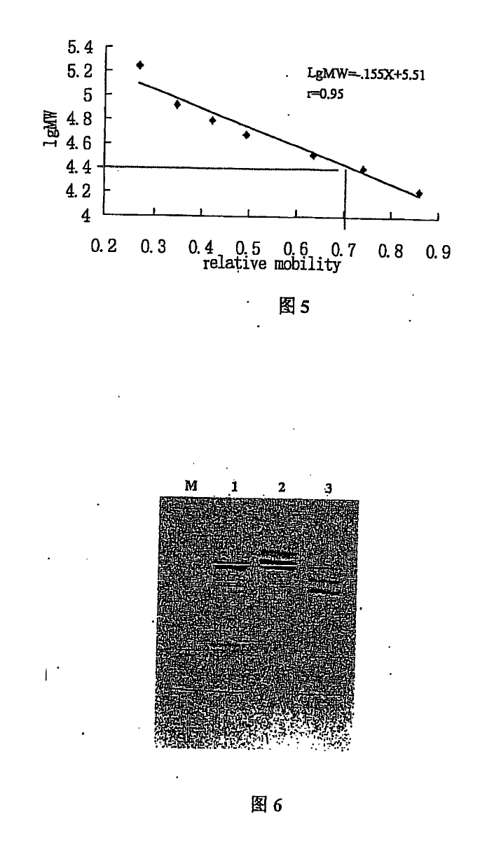

Results: The single peak was obtained (FIG. 3). Its fibrinolytic activity was 90.49±12.41 mm²/μg.

### EXAMPLE 2

The determination of purity and biological characterization for the fibrinolytic enzyme FII from Agkistrodon acutus venom 2.1 Purity determination of the fibrinolytic enzyme FII.

Method: Sodium dodecyl sulfate-polyacrylamide gel electrophoresis (SDS-PAGE) was performed according to the method of Laemmli (12% resolving gels, pH 8.8; 4% stacking gels, pH 6.8; Tris-glycine as the electrophoresis buffer composition; voltage, 200 v; time, 45 minutes) Molecular weight standards were MBP-β-galactosidase (175,000), MBP-paramyosin (83,000), Glutamic dehydrogenase (62,000), Aldolase (47,500), Triosephosphate isomerase (32,500), β-Lactoglobulin A (25,000), Lysozyme (16,500). The protein bands were stained with coomassie brilliant blue R-250 solution.

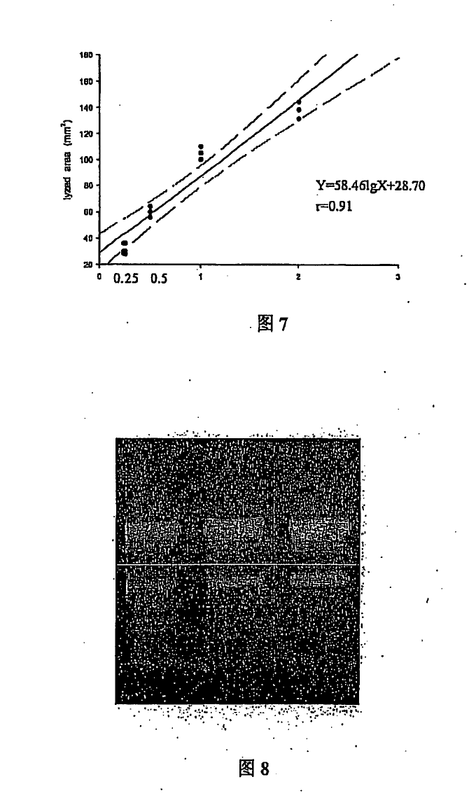

Results: On the SDS-PAGE gel, the purified FII appeared as a single protein band (FIG. 4). The equation, 1 gMW=−1.55+5.51, r=0.95, was obtained according to the regression curve (X-axis, 1 gMW; Y-axis, the relative mobility of standard mobility (Rf(x))). Its calculated molecular weight is 25,550 (FIG. 5). 2.2 Fibrinogenolytic activity determination of the fibrinolytic enzyme FII from Agkistrodon acutus venom.

Method: Bovine fibrinogen (75 μL, 0.2 mg/mL) was pipetted into 1.5 mL microcentrifuge tubes. FII (25 μL, 2 mg/mL) was added to the tube and the solution was incubated at 37° C. for 1 hour. Then the reaction in the tube was stopped by the addition of 10 μL of 125 mM EDTA followed by 50 μL of SDS-PAGE sample buffer. 20 μL of sample was applied for SDS-PAGE analysis to determine whether FII degradation of fibrinogen varied from that of plasmin degradation, the same experiment was repeated except for plasmin in place of FII.

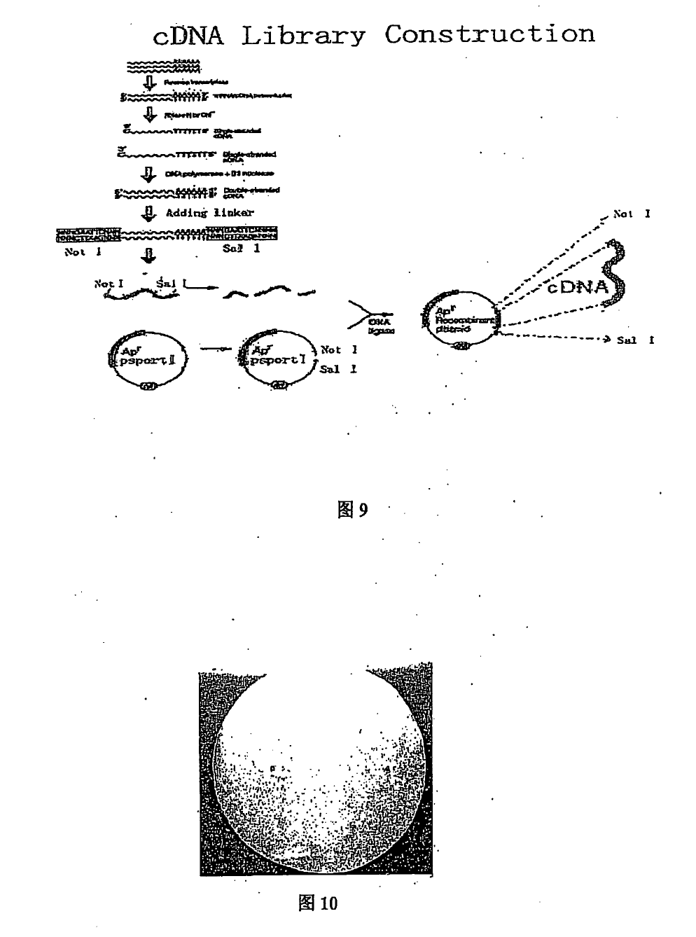

Results: SDS-PAGE showed FII is an α,β-fibrinogenase with the α,β chains being degraded, while the γ chain appeared unaffected. The molecular weight of the degradation fraction by FII was 45,000 Da. The α, β and γ chains of fibrinogen were degraded by plasmin. The molecular weight of the degradation fractions by plasmin was 47,000 Da, 44,000 Da, 23,000 Da, respectively. The bands after degradation by FII and plasmin were different, which indicate they have different cleavage sites (FIG. 6). 2.3 Fibrinolytic activity determination of fibrinolytic enzyme FII from Agkistrodon acutus venom.

Method: Fibrinolytic activity was demonstrated using a modified fibrin plate technique (Astrup and Mullerti, 1952). Bovine Fibrinogen (0.2%, 20 mL) was clotted in a Petri dish (diameter, 9.5 cm) with thrombin (80 μL, 100 U/mL). Each concentration was tested three times. Fribrinolytic activity was expressed as the diameter product (mm2) of the lysed zone caused by 20 μL of each fraction. Each fraction was put on the surface of the dish and incubated at 37° C. for 12 hours. Urokinase 500 U/mL was used for positive control and the saline solution was used for the negative control.

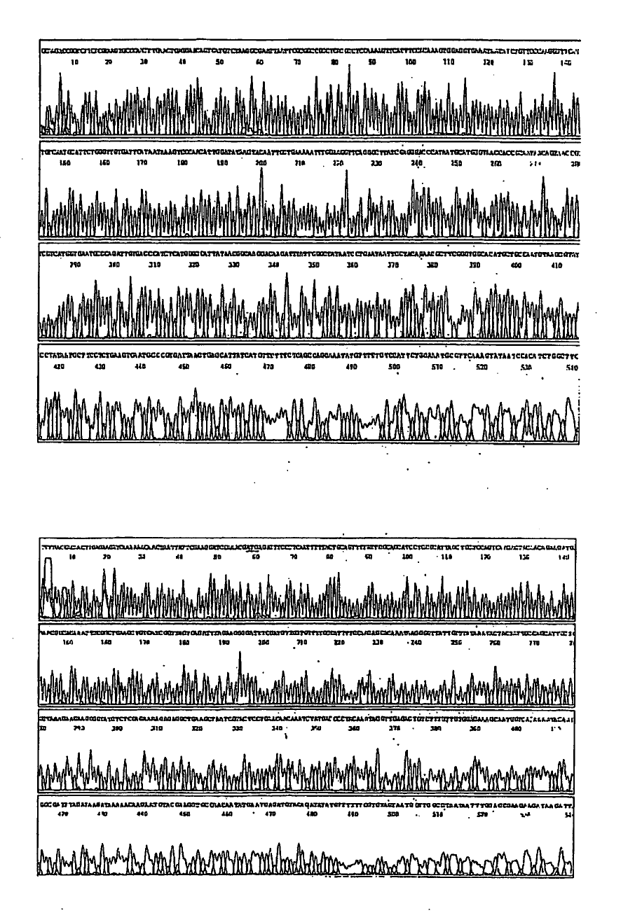

Results: FII, at 20 μL at several different concentrations, 0.25, 0.5, 1 and 2 mg/mL was applied (table 1). It was shown that the lysed areas increased with the increased concentration (FIG. 7).

TABLE 1
The fibrinolytic activity of FII (n = 3, mean ± SD)

| Concentration | Lysed area |
|---|---|
| (mg/mL) | (mm²) |
| 0.25 | 31.33 ± 3.40 |
| 0.5 | 60.00 ± 3.27 |
| 1 | 105.00 ± 4.08 |
| 2 | 137.67 ± 5.31 |

### EXAMPLE 3

Preparation of the antibody of fibrinolytic enzyme FII from Agkistrodon acutus venom 3.1 Preparation of FII antiserum of rabbit

Method: (1) Animal immunity. Rabbits were immunized by multi-point subcutaneous injection at the back with the emulsifier mixed with 1 mL of FII (3 mg/mL) and 1 mL Freund's adjuvant. After the first injection, the subsequent boosters were given per week, totally 4 weeks.

(2) Preparation of antiserum. The immunized rabbits were anaesthetized by an intramuscular injection of 30 mg/kg ketamine hydrochloride. Blood samples were taken through a catheter inserted into a Carotid and stored at 4° C. overnight. The sera were subsequently separated by centrifugation and aliquots were stored at 4° C.

(3) Determinations of antibody titer by enzyme-linked immunosorbent assay (ELISA). Microtiter plates (96 wells) were coated overnight at 4° C. with 100 mL (0.1 mg/mL) of FII in sodium bicarbonate buffer, 100 μL per well and was stored at 4° C. overnight. The plates were washed two times with PBST containing 0.05% Tween-20 and unbound sites were blocked for 1 hour at 37° C. with blocked liquor containing 2×PBS, 1% bovine serum albumin (BSA). To measure the serum titers, 100 ml of serial dilutions of serum in 1×PBS containing 1% BSA and a rabbit antiserum (primary antibody) were added to the plates and incubated for 1 hour at 37° C. The plates were washed three times again and incubated for 1 hour with 100 mL of a goat anti-rabbit immunoglobulin G (whole molecule)—peroxidase conjugate, followed by further washing for three times. The substrate solution for the peroxidase assay A and B were added into every well, the antibody titer was identified according to color of reaction system.

Result: No. 1 and 2 rabbits were immunized by the Purified fibrinolytic enzyme FII from Agkistrodon acutus venom using as antigen. After 4 weeks, antibody titer was determinated by ELISA. The antibody titer was 1:20000 in each rabbit. 3.2 Preparation of FII monoclone antibody.

Method: (1) immunity, hybridoma Fusion and cloning screening. Balb/c mice (7-week old) were subcutaneously injected with a mixture containing 0.2 mL (1 mg/mL) FII and an equal volume of Freund's complete adjuvant. A boost injection with the same amount of antigen in Freund's complete adjuvant was administered at 2-week intervals, totally 3 cycles. 0.2 mL (1 mg/mL) FII was injected through vena caudalis. 3 days later, hybridoma fusion was performed. In brief, the splenocytes were harvested from immunized mice, mixed with SP2/10 cells at a 10:1 ratio, and fusion was carried out with 50% PEG (u 3700). The fused cells were cultured in 5 96-well plates, and cultured in a 5% CO2 incubator. HAT selective medium including 15% FBS were consecutively applied for 4 days, followed by HT medium for 6 days, from the eleventh day on, RPMI-1640 medium including 10% FBS was kept in using.

(2) Antibody produced in medium was measured by indirect enzyme-linked immunosorbent assay (indirect ELISA) as described above.

Result: 20 cell lines were established after 2 cycles of hybridoma fusion. Among which, 2 cell lines with higher specificity were named C1 and C4, respectively. The monoclone antibody produced by the two cell lines all were IgG I, testing by IgG typing kit, used according to the instructions of the manufacturer.

### EXAMPLE 4

Construct an expressing cDNA library from Agkistrodon acutus venom gland 4.1 Preparation of RNA.

Method: (1) The Agkistritrodon snake was sacrifice by decapitation. The pairs of venom glands were extracted from the snakes' heads and stored in solid carbon dioxide immediately.

(2) The total RNA was extracted by using the RNA purification kit (Promega). The venom gland (for a total of about 1.7 g) was homogenated, then mixed with 20 ml apomorphosis solution and homogenated 20 seconds again. Sodium acetate anlydrous (2M, 2 ml) was mixed fully with the homogenation, then trizol, chloroform and isopropyl alcohol were used respectively to homogenize the gland, shaked for 10 seconds, iced bathed for 90 seconds, centrifuged for 20 minutes at 12,000 rpm. The supernatant was mixed with the equal volume of isopropyl alcohol, and was stored in −30° C. for 20 min, then centrifuged in 4° C. for 15 minutes at 12,000 rpm, separated the aqueous phase of RNA, deposit the total RNA with isopropyl alcohol. The RNA pellet was washed twice with 75% iced ethanol and briefly air dried. In the end, the RNA was dissolved in diethylpyrocarbonate (DEPC) treated H₂O. The integrity of total RNA was checked by discerning the 28 S and 18 S bands of ribosomal RNA in 1% agarose gel. The purity of the total RNA was checked by the ratio of A260/A280.

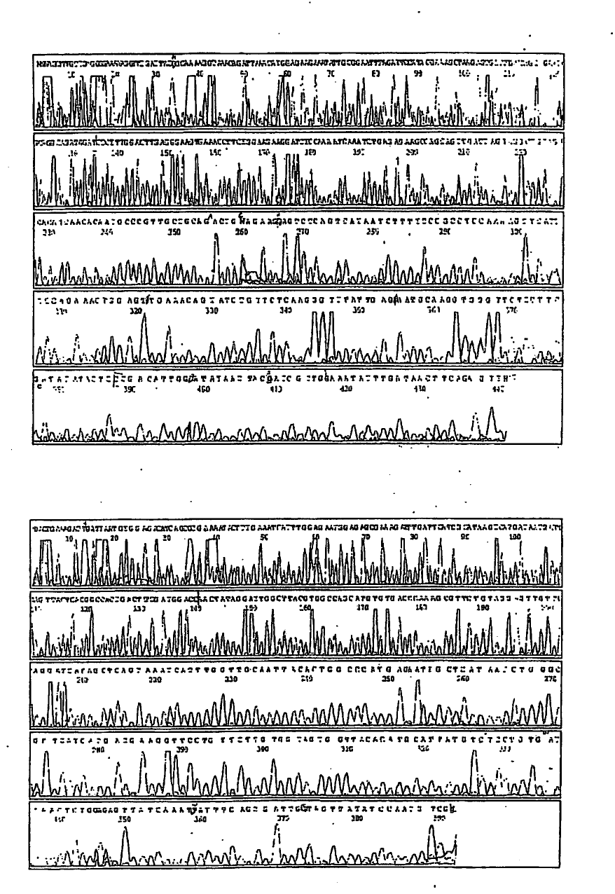

Result: the total RNA appeared as a long smear with clear bands of 28 S and 18 S. The ratio of OD260/OD280 to the total RNA was 20, and the total amount was 426 μg. So the conclusion can be drawn that the RNA didn't degrade and that the quality is high. RNA electrophoris (FIG. 8) show 28 S and 18 S rRNA clearly and the fluorescence intensity of 28 S was two times that of 18 S rRNA, indicating a foundation for a high quality library. 4.2 Isolation and purification of mRNA.

Method: mRNA was subsequently purified by means of oligo (dT)-cellulose affinity chromatography, following its manufacturer protocol. Poly (A) RNA was enriched from total RNA: sample buffer solution sufficiently wash the cellulose column, the total RNA (3 mg) in DEPC treated H2O was incubated at 65° C. for 5 minutes. Decrease temperature to room temperature quickly by means of iced bath. The total RNA was applied to a column of oligo (dT)-cellulose affinity chromatography eluted with sample buffer solution for three times, followed by eluant, mRNA was washed and isolated.

The total amount of purificated mRNA was determinated by testing the absorbance of mRNA at 260 nm.

Result: The total amount of mRNA was 10 μg.

4.3 Construction of a cDNA Library

Method: Using the cDNA synthesis kit (Promega) and following its manufacturer protocol. See (FIG. 9)

1) Synthesis of the first strand cDNA

(2) Synthesis of the second strand cDNA

(3) Adding Not I and Sal I adaptors at the end of cDNA

(4) Ligation and transformation of cDNA cDNA and pSport I vector were ligated. The reaction mixtures of cDNA, pSport I vector (1 μg), T4 ligase 10× buffer, T4 DNA ligase were incubated at 16° C. overnight. 10 μL of product of ligation was added into 50 μL competence E. coli DH5, in order, 4° C., 30 minutes, 42° C. 120 seconds, 4° C., 5 minutes, 37° C., 10 minutes. Then the suspended cells were then placed into a new 1.5 mL EP tube and rotated at 37° C. for 1 hour under 250 rpm. 2 μL of transformation product were diluted 10, 100 and 1000 times and plated on Luria-Bertani plates containing 50 μg·mL⁻¹ of ampicillin with isopropy-β-D-thiogalactoside and 5-brom-4-chloro-3-indolyl-beta-D-galactopyranoside. Plates were incubated at 37° C. overnight. The titer of the library was calculated and stored the library at −80° C.

Result: The total titers of five unamplified libraries are 5×10⁷ pfu/mL.

### EXAMPLE 5

Clone of FII Gene From Expression cDNA Library

5.1 Screening Expression cDNA Library with Antibodies

Method: (1) after proper dilution, 100 L bacterium in cDNA library were spreaded into plates containing LB medium supplemented with 1% glucose, 8% glycerol and 50 μg/mL ampicillin and grown 12-16 h at 37° C. till the colonies was got needle size.

(2) Cellulose acetate membrane was soaked with IPTG (10 mmol/L) and spreaded on the surface of LB plates covered with colonies, three asymmetrical spot was left on the surface of cellulose acetate membrane to facilitate orientation. Cellulose acetate membrane was unveiled and spreaded on the surface of agarose plate (the side covered with colonies is upward), plates were incubated at 37° C. for 6˜8 hours for induction of fusion protein expression. The LB plate was stored in 4° C.

(3) After 6˜8 hours of induction, cellulose acetate membrane was treated with chloroform for 30 minutes. The membrane was subsequently blocked overnight at 4° C. in 1% BSA, 4 μg/mL lysozyme and 1 μg/mL DNAase with gentle agitation for 30 minutes. After blocking, filters were washed three times for 15 minutes per wash in PBST. To detect colonies producing recombinant protein, the membrane was probed with rabbit-antibody at a 1:1000 dilution and 1% BSA in PBS buffer with gentle agitation for 3 hours at room temperature, The membrance were washed three times again and incubated for 3 hour with 100 mL of a goat anti-rabbit immunoglobulin G (whole molecule)-peroxidase conjugate, followed by further washing for three times.

The membrane was soaked in DAB colouration solution with gentle agitation for 30 minutes, rinsed with distilled water, positive colonies with brown color were detected.

Those positive colonies were spreaded in LB plates respectively and screened with monoclone antibody by the method of the above described.

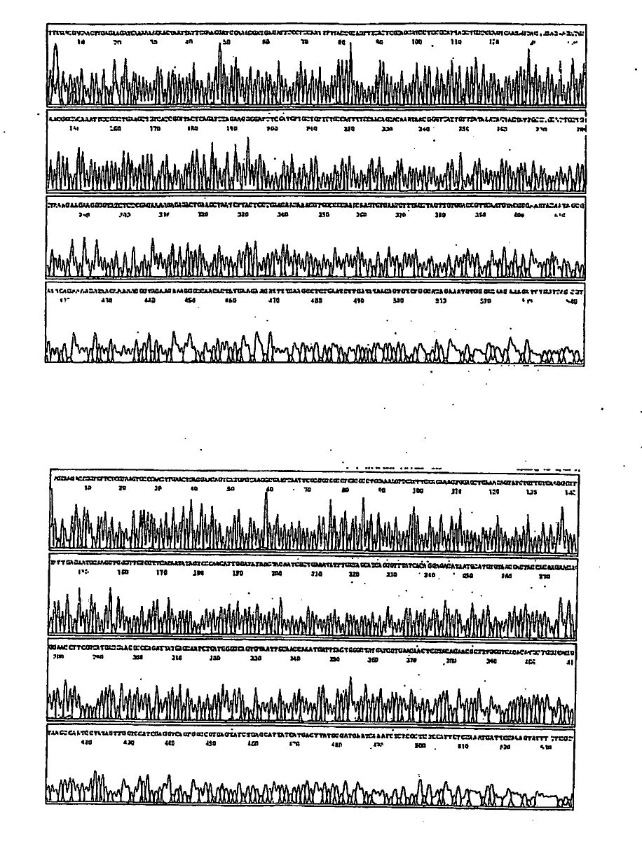

Results: primary screening was performed with plates (9 cm in diameter, including 5×103 clones). 200,000 clones were screened and 15 positive colonies were found. Repeated screening was performed in those positive clones and positive reactions were observed in 11 clones (FIG. 10). Those 11 positive clones were plated on Luria-Bertani plates containing 100 mg L-1 of ampicillin, detected with monoclone antibody, positive reactions (C1 and C4 antibody were used) were observed in 9 clones.

### 5.2 Plasmid Extraction and Sequencing of Positive Clone

Method: the plasmids were extracted by alkaline lysis (QIAGEN). The positive clonies were cultured at 37° C. for 24 h in 5 ml LB media with shaking, respectively.

(1) Take 1.5 ml overnight cultured bacterial solution, and the bacterial cells were harvested by centrifugation at 1,200 rpm for 30 seconds at 4° C.

(2) discarded the supernatant, The bacterial pellet was resuspended in 0.25 mL of Buffer P1. Shake vigorously until the bacterial pellet was dispeared.

(3) 0.25 mL of Buffer P2 was added, mixed thoroughly by vigorously inverting the sealed tube 5 times.

(4) 0.35 mL of chilled Buffer N3 was added, mixed immediately and thoroughly by vigorously inverting 5 times so that the buffer was uniform dispersion in viscious creaking of bacteria.

(5) Centrifuged at 12,000 rpm for 10 minutes at 4° C. Whit desposition could be fould.

(6) The supernatant was moved to a QIA column and centrifuged for 30-60 seconds. Discarded the centrifugate solution.

(7) Added 0.5 ml buffer PB and centrifuged for 30-60 seconds. Discarded the centrifugate solution.

(8) Added 0.75 ml buffer Pe and centrifuged for 30-60 seconds. Discarded the centrifugate solution.

(9) centrifuged for 1 min without adding any buffer.

(10) Added 50 μl buffer EB or H₂O, put quescently 1 min, then centrifuged for 1 min.

The extracted plasmids were used for capillary sequencing applying the standard primers T7 and SP6 by the method of Sanger Dideoxy termination.

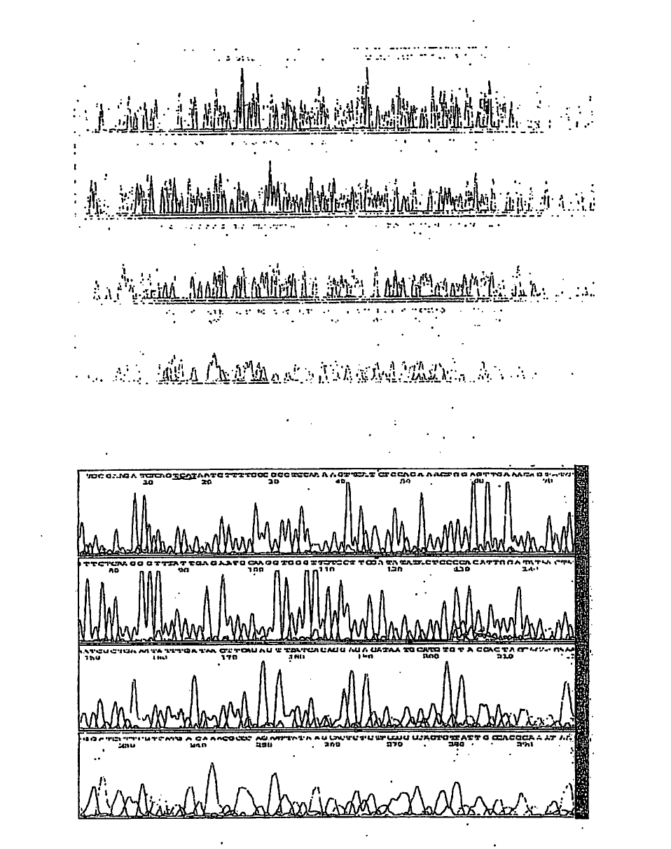

Result: A single colony was picked from the 9 positive plates respectively and a starter culture of 2-5 mL LB medium containing the appropriate selective antibiotics was inoculated, then incubated overnight at 37° C. with vigorous shaking for extracting plasmid. PCR reaction was performed applying the standard primers T7 and SP6. Specifical bonds of product were observed in each clone. The plasmids were used for capillary sequencing by the standard primers T7 and SP6 (FIG. 11).

### 5.3 Sequence Homology Analysis

Method: In order to identify the genomic origin of those positive clones, the programs BLASTn and BLASTp obtained in the web site of NCBI (The National Center of Biotechnology Information) was used to conduct sequences and protein homology to determine the plasmid having the genomic origin of FII.

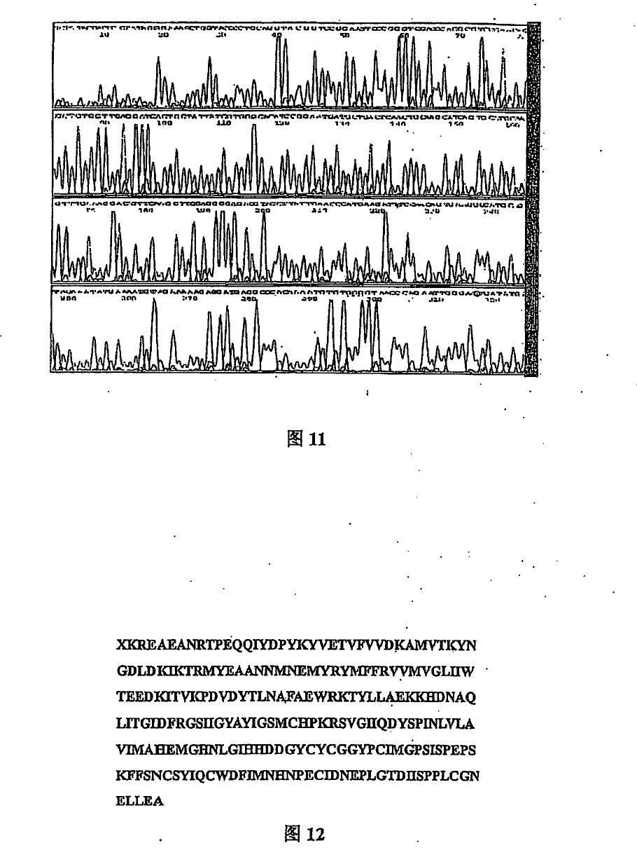

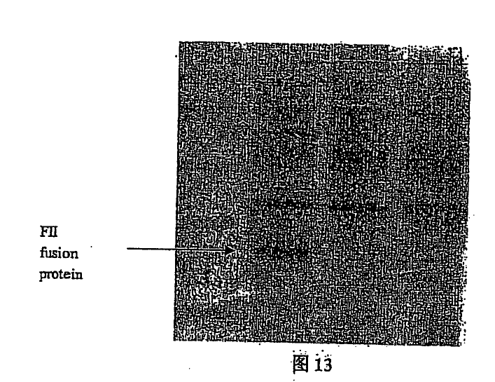

Result: Compared with the homology in the NCBI-BLASTn and BLASTp database, most of those 9 sequences were homologous to the SVMPs family (sharing homology to reported gene with the highest identities of 85%). Among which, FII-2 and FII-4 share the most conservative sequences, belonging to ADAM family, which have relationship with SVMPs family. So, the gene of FII-2 was named as FII and the protein was preferentially expressed, and the amino acid sequences of FII is deduced (FIG. 12).

### EXAMPLE 6

Expression of FII Gene from Agkistrodon acutus venom in E.Coli.

Method: 0.2 mL of E.Coli (colonies containing FII plasmid confirmed by extraction) was added to SOB medium containing 20 mL of ampicillin and shaked in 37° C. for 3 hours until A600 was up to about 0.5. IPTG was added to the final concentration of 1 μmol/L, then shaked for 4 hours and centrifuged at 1,500 rpm for 20 minutes, and the supernatant was discarded. 0.5 mL of water and 0.5 mL of SDS loading buffer was added to the pellet, then 100° C. bathed for 5 minutes and centrifuged at 10,000. 20 μL of supernatant was taken to load for SDS-PAGE.

Result: Lysate of E.Coli containing FII plasmid after induced by IPTG showed a band of 40 kD in SDS-PAGE, while those of E.Coli without IPTG induction or without plasmid did not show such a band, indicating that the gene of FII can be expressed as fusion protein in E.Coli.

### EXAMPLE 7

Cloning of FII gene of from Agkistrodon acutus venom.

Method: (see FIG. 14)

### 7.1 PCR Amplification of FII

The sequence of α signal to 5′ of target gene was introduced by two-step PCR and the restriction end nuclease site as well.

Primer 1:
5′AGA GAG GCT GAA GCT AAT CTT ACT CCT GAA C3′
Primer 2:
5′CT CTC GAG AAA AGA GAG GCT GAA GCT AAT C3′
Reverse primers:
Primer 3:
5′GAG CGG CCG CCT CAC GCC TCC AAA AGT TC3′

### Procedure:

(1). The first round PCR: forward primer is primer 1, and reverse primer is primer 3; the template is FII plasmid.

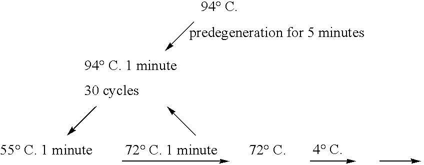

(2) The second round PCR: forward primer is primer 2, and reverse primer is primer 3; the template is the production of the first round of PCR. And the PCR condition is the same with the first round PCR.

7.2 Construction of Recombinant PPIC9-FII plasmid

The production of the second round PCR was extracted after electrophoresis on 1% agarose gel. The target fragment extracted from agarose gel and PPIC-9 plasmid were digested with XhoI and NotI, and retrieved after electrophoresis, respectively; then connected in T4 reaction system for 12 hours in 16° C., and subsequently transformed to competent Top 10 F cells and plated on LB/penicillin plate. Positive colonies were picked up, plasmid were extracted and identified by restriction endonuclease analyses, and then the plasmid containing target fragment were purified.

### 7.3 Construction of Recombinant Expression PPIC9K-FII Plasmid

PPIC9K-PPIC9 plasmid with or without target fragment were digested both with SacI and NotI, connected and transformed. And then positive colonies were picked up, PPIC-9K-FII plasmid were identified and purified.

Result: After the above two-step PCR, the PCR product was about 700 bp and was the same large with the gene of FII. The retrieved PCR product after electrophoresis and PPIC9K plasmid were digested both with XhoI and NotI, and then constructed recombinant PPIC9K-FII plasmid identified by restriction endonuclease digestion.

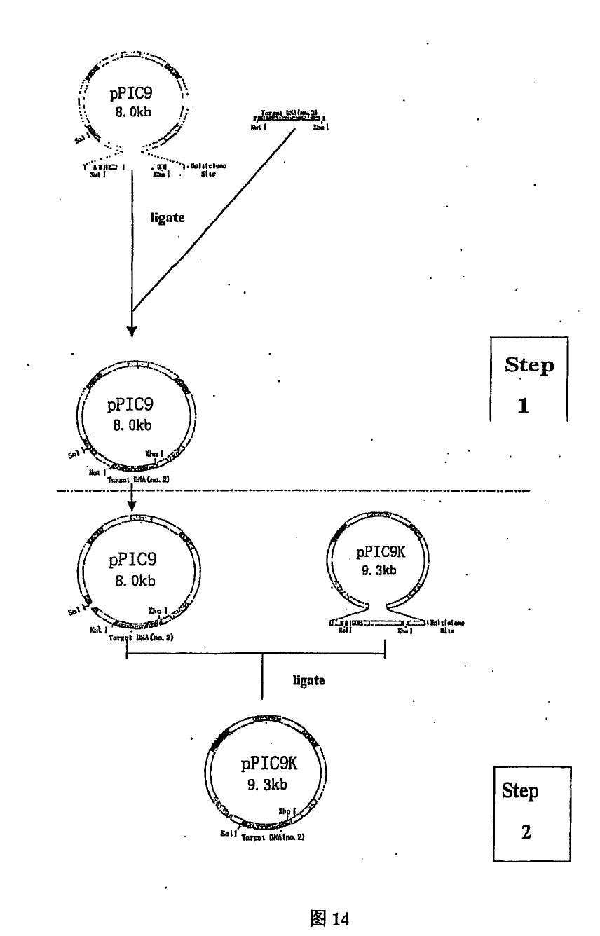

Recombinant PPIC9K-FII plasmid both with SacI and NotI were digested, and subcloned the little digestion fragment to PPIC9K plasmid also digested both with SacI and NotI to construct recombinant expression PPIC9K-FII plasmid. The product identified by restriction endonuclease digestion was 700 bp and was the same with anticipation (see FIG. 15). Sequencing by Sanger method showed that the DNA sequence of FII did exist in recombinant expression PPIC9K-FII plasmid.

### EXAMPLE 8

Expression of the recombinant fibrinolytic enzyme FII from Agkistrodon acutus venom in yeast.

### 8.1 Transformation and Screening of Yeast

Method: 50 μg of recombinant plasmid PPIC9K-FII was isolated from the E.coli and linearized at the SacI restriction enzyme site, followed by extraction with phenol and chloroform, and precipitation with ethanol. After that, the DNA was dissolved in TE buffer and stored at −30° C. The linear plasmid was mixed with the salmon sperm DNA and was added to competent yeast prepared with 3% polyglycol, which was plated to two RD plates well-distributedly. The plates grew at 30° C. upside down. After 96 hours, the yeast strains were washed down the RD plates and plated on YPD plates containing a variety of G418 concentrations including 0.5, 1, 2, 3, 4 mg/mL. The YPD plates grew at 30° C. upside down. Recombinant colony first showed up in the plate with low G418 concentration on the fourth day. On the 6th day, colonies from plate with 3 mg/mL of G418 were selected to isolate DNA for PCR analysis.

Results: plasmid PPIC9K-FII was isolated and linearized at SacI site. The linear plasmid was purified and transformed into competent yeast with PEG method. The first colony showed up in YPD plate with G418 low concentration (0.5 mg/mL) on day 4, and on day 6, 6 colonies were selected from the plate with 3 mg/mL G418.

### 8.2 Screening for High Level Expression Strain and Scale-up Expression of Recombinant FII

Method: 6 recombinant strains that have been confirmed by PCR to contain our insert genes were inoculated in 10 mL of MD media respectively, cultured at 30° C. overnight with shaking. Then the cells were harvested by centrifugation, and added into BMMY media to induce expression. 100% methanol was added to a final concentration of 1% methanol every 24 hours to maintain induction. Sampling was also performed every day from the induction cultured and reacted with fibrinogen at 37° C., followed by SDS-PAGE, to analyze its fibrino(gen)lytic activity. 3 tubes of samples were quickly frozen on the 3rd day.

A tube of frozen strain was selected to 100 mL MD media and cultivated at 30° C. with shaking overnight and amplified to 2 L culture in two 5 L flasks respectively. After 24 hours, the cultures was transfered to BMMY media in jar fermenter of 100 L volume and continued to grow at 30° C. And supplement with 1% methanol every 24 hours. After 96 hours fermentation, the culture was centrifuged and the supernatant was saved, which was later ultrafiltrated into 2 L.

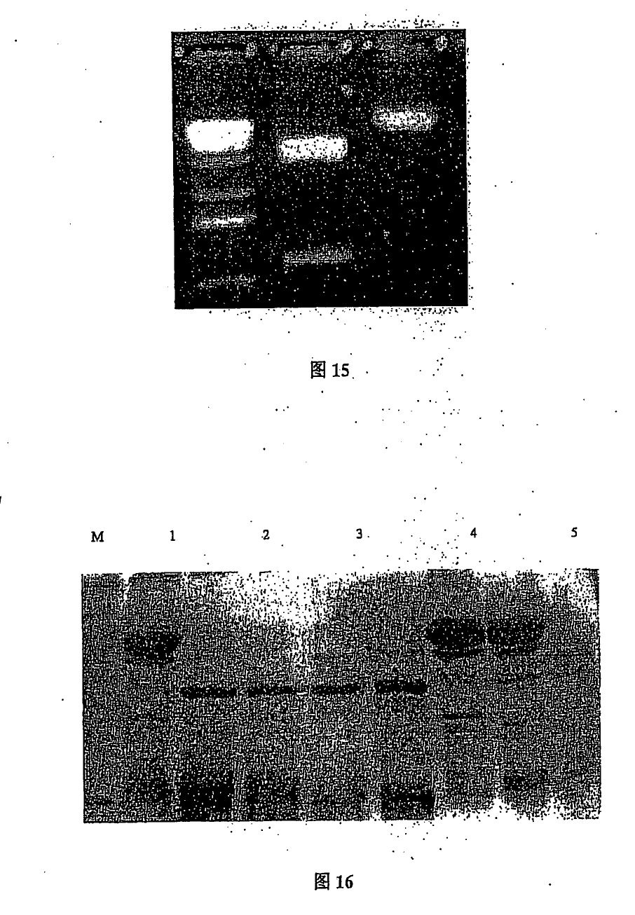

Results: 6 recombinant strains which have been confirmed to contain FII gene was cultivated. Sampling was done every day and its supernatant was used to react with fibrinogen at 37° C. for 1 hour, and subjected to SDS-PAGE analysis. The 3rd day samples from number 1, 2, 3 yeast strains have showed hydrolysis activity towards fibrinogen (FIG. 16), natural FII was used as a positive control. The recombinant FII is identical with natural FII in terms of hydrolysis modality and dominant fraction after hydrolysis, which suggested that these three yeast strains are capable of expressing recombinant FII in high efficiency. Large scale recombinant FII can be obtained in culture media supernatant after 96 hours fermentation with methanol induction.

### EXAMPLE 9

Purification and biochemical characterization determination of the recombinant fibrinolytic enzyme FII from Agkistrodon acutus venom.

### 9.1 DEAE-Sepharose FF Negative Ion Exchange Chromatography

Method: (1) ultra filtration. The supernatant liquor was ultra filtrated by Millipore until the column was is 2 L, double column of 0.05 M NH4Ac was added and continued to ultra filtrate.

(2) DEAE-Sepharose FF. The supernantant ultrafiltered was applied to a column of DEAE-Sepharose FF previously equilibrated with 0.05 M NH4Ac (pH 8.0). The column was washed with 0.1 M NH4Ac (pH 6.5) to remove unbound material and the remained proteins were then eluted with 0.2 M NH4Ac (pH 5.2).

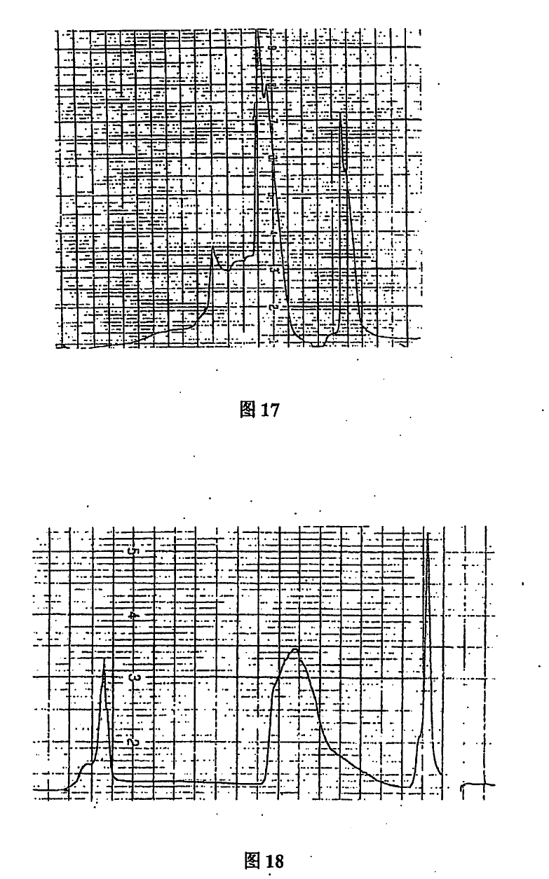

Results: The supernantant ultrafiltered on DEAE-sepharose yielded 5 fractions (FIG. 17). The fibrinolytic activity was localized in the fraction in the 0.2 M NH4Ac (pH 5.2).

### 9.2 Hydrophobic Chromatography

Method: The fraction displaying fibrinogenolytic activity was then mixed with 2 M NaCl and applied to another column of Butyl-toyopearl 4 FF previous equilibrated 1 M NaCl, 20 mM PBS. The column was washed with 1 M NaCl, 20 mM PBS to remove unbound material and the remained proteins were then eluted with 2 mM PBS.

Results: The supernantant ultrafiltered of the fraction on Butyl-toyopearl gave 3 fractions (FIG. 18). The fibrinolytic activity was localized in the third fraction. Recombant FII was obtained by ultrafiltering, purifying from salts and lyophilizing the fraction displaying fibrinogenolytic activity.

9.3 Purity Determination of the Recombinant Fibrinolytic Enzyme FII from Agkistrodon acutus venom

Method: The Purity determination of recombinant FII was performed by the method of SDS-PAGE mentioned as description in methods 2.1.

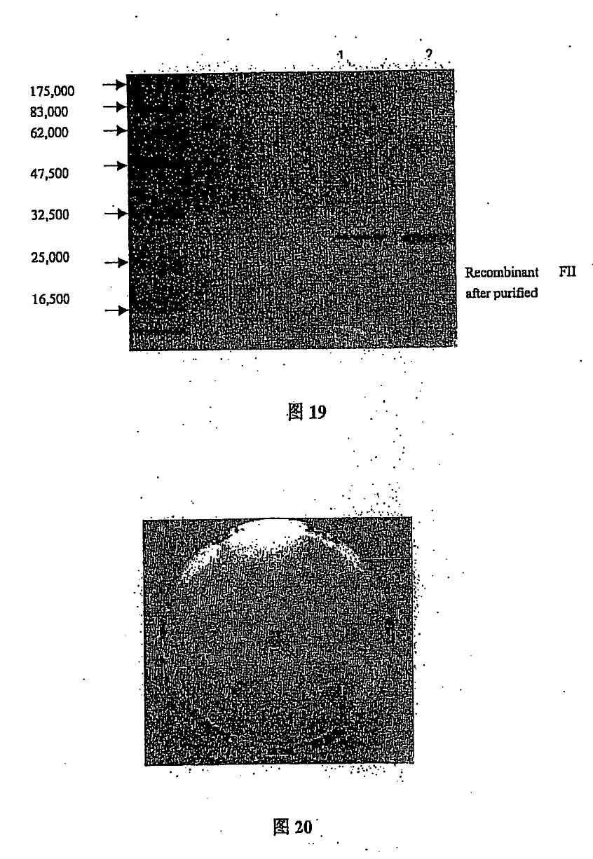

Results: On the SDS-PAGE gel, the purified recombinant FII appeared as a single protein band (FIG. 19)

9.4 The Directly Fibrinolytic Activity Determination of the Recombinant Fibrinolytic Enzyme FII from Agkistrodon acutus venom

Method: The fibrinolytic activity of FII was demonstrated using a heated fibrin plate which was incubated at 85° C. for 30 minutes in order to inactivate the plasmin activity. 20 microliters of different concentration of recombinant FII (2 mg/mL, 1 mg/mL, 0.5 mg/mL, 0.25 mg/mL, 0.125 mg/mL), were applied onto the prepared plate surface. Nature FII 0.5 mg/mL and urokinase 500 U/mL were used for positive control and the saline solution was used for the negative control. Each concentration was tested 3 times. The plate was incubated in a humid chamber at 37° C. for 12 hours. Fibrinolytic activity was measured by the area of the clear lysed zone.

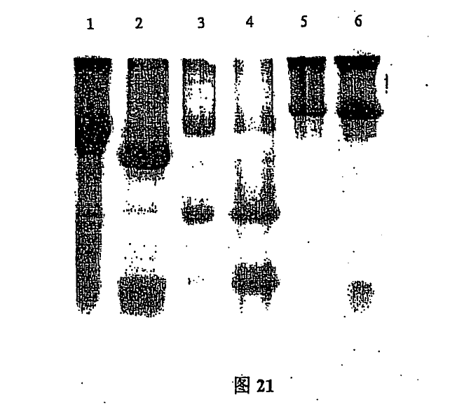

Results: The fibrinolytic activity of FII was demonstrated using a heated fibrin plate technique. It was shown that rFII could formed clear lysis zone on the fibrin plate with increasing concentration, its fibrinolytic activity was higher than nature FII (the lysed zone by 0.5 mg/mL rFII was 50 mm2, and the lysed zone by 0.5 mg/mL nature FII was only 40 mm2) (Table 2, and FIG. 20). The plasmin was inactivated when the fibrin plate was heated to 85° C.; it was showed that recombinant FII could dissolve fibrin without depending on the plasminogen activation.

TABLE 2
The directly fibrinolytic activity of the recombinant FII

| Concentration | Lysed area |
|---|---|
| (mg/mL) | (mm²) |
| 2 | 147 |
| 1 | 110 |
| 0.5 | 50 |
| 0.25 | 28 |
| 0.125 | 14 |
| Natural FII 0.5 | 40 |

### 9.5 The Effect of Recombinant FII on Human IgG and Albumin.

Method: Human IgG (10 μL, 10 mg/mL) mixed with r-FII (10 μl, 0.5 mg/mL) was incubated at 37° C. for 24 hours using a pipette. After the indicated time, 10 μL Protein electrophoresis Solution was added. The sample was analyzed by SDS-PAGE. The same experiment was repeated except for human albumin in place of human IgG.

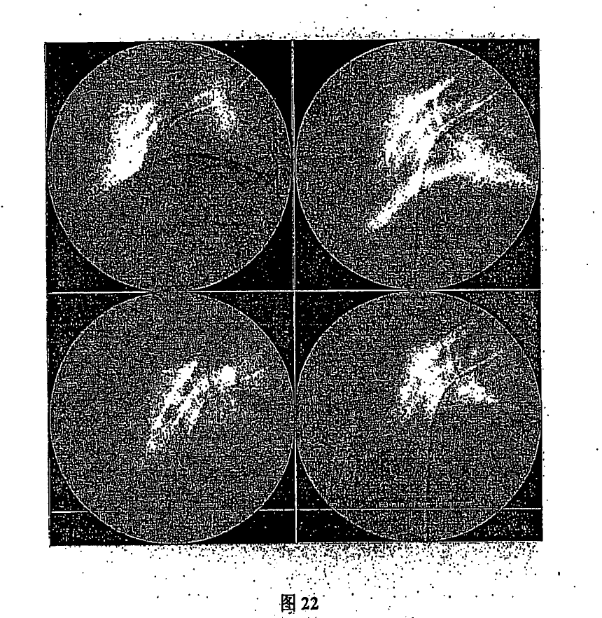

Results: When IgG was incubated with recombinant FII, the heavy chain disappeared, while the light chain was still insusceptible to the enzyme. When albumin was incubated with recombinant FII, the peptide chains almost remained integrity. It suggested that recombinant FII have no effect on albumin (FIG. 21).

### EXAMPLE 10

The effect of recombinant FII on the Beagle's Pulmonary Artery thrombosis.

Method: Beagle's Pulmonary Artery thrombosis experimental models were performed by the method of Nowk. The blood clot was prepared by incubating the 15 mL experimental Beagle's whole blood at 37° C. for 2 hours in vitro before application. Two different groups were established, containing 6 animals each: the treatment group was given 0.24 mg/kg rFII. The NS control group was infused with 0.1 ml/kg NS. Animals were anaesthetized by an intramuscular injection of 30 mg/kg pentobarbital sodium, followed by intramuscular supplements of ketamine hydrochloride given throughout the experiment. The left femoral vein was separated from surrounding tissue after anesthesia. A 6-mm2 Fcobra/Hunterhead catheter sheath was inserted into the left femoral vein, through the infrarenal abdominal vein, the right atrial, the right ventricular, the pulmonary trunk, and was finally introduced in the right lower pulmonary artery. The artificial thrombus was injected into the catheter positioned in the right lower pulmonary artery. Selective intra-arterial digital subtraction angiography (DSA) was performed on a biplane high-resolution angiography system with a matrix of 1024 cm×1024 pixels to record the thromboembolism in the right lower pulmonary artery. Two hours after the infusion of the preformed thrombus, single dosages of recombinant FII were injected through the femoral vein. The recanalization rate of thrombosis of the right lower Pulmonary artery was measured by the lateral and auteroposterior (AP) projection images before rFII infusion and at 15,30,60,90 and 120 minutes post-infusion to evaluate the effect of r FII on artery thrombosis.

Results: Measurements made at 1 hour after recombinant FII infusion showed that recombinant FII efficiently dissolve the preformed thrombus in the right lower Pulmonary artery (FIG. 22). The average of the recanalization rate of thrombosis in the right lower Pulmonary artery was 83.3%. Little changes were detected in the NS control groups measured 1 hour after infusion. The average of the recanalization rates of thrombosis in the right lower Pulmonary artery was 0.6%.

TABLE 3
The thrombolytic effect of recombinant FII on the thrombosis
of Beagle's right lower pulmonary artery

| Beagle dog | | Recovery rate of pulmonary artery |
|---|---|---|
| (number) | Time after injection | (%) |
| 3 | 1 h | >90 |
| 2 | 1 h | >80 |
| 1 | — | >70 |
| 6 (0.9% NaCl) | 1 h | 0.6 |

1. An isolated polynucleotide comprising SEQ ID NO: 1.

2. The isolated polynucleotide of claim 1, wherein the sequence is a DNA sequence.

3. A vector comprising the DNA sequence of claim 2.

4. A genetically engineered host cell comprising the vector gene of claim 3.

5. A pharmaceutical composition for dissolving fibrin(ogen) and treating thromboemolic disorders, the pharmaceutical composition comprising the isolated polynucleotide of claim 1.

6. A pharmaceutical composition for dissolving fibrin(ogen) and treating thromboemolic disorders, the pharmaceutical composition comprising the isolated polynucleotide of claim 2.

7. An isolated polynucleotide sequence that has at least about 80% sequence identity to the isolated polynucleotide sequence of claim 1.

8. An isolated polynucleotide sequence that has at least about 85% sequence identity to the isolated polynucleotide sequence of claim 1.

9. A pharmaceutical composition for dissolving fibrin(ogen) and treating thromboemolic disorders, the pharmaceutical composition comprising the isolated polynucleotide of claim 7.

10. A pharmaceutical composition for dissolving fibrin(ogen) and treating thromboemolic disorders, the pharmaceutical composition comprising the isolated polynucleotide of claim 8.
